(19) United States
(12) Patent Application Publication
(10) Pub. No.: US 2026/0217851 A1
(43) Pub. Date: Jul. 30, 2026

(54) Compositions that Target CD138 and CD3 and Methods of Making and Using the Same

(71) Applicant: The Medical College of Wisconsin, Inc., Milwaukee, WI (US)

(72) Inventors: Charles E. Hay, Wauwatosa, WI (US); Jeffrey A. Medin, Shorewood, WI (US)

(21) Appl. No.: 19/141,719

(22) PCT Filed: Dec. 22, 2023
(86) PCT No.: PCT/US2023/085729

§ 371 Date: Jun. 20, 2025

Related U.S. Application Data
(60) Provisional application No. 63/476,766, filed on Dec. 22, 2022.
(60) Provisional application No. 63/476,762, filed on Dec. 22, 2022.

(51) Int. Cl. C07K 16/28

(52) U.S. Cl. CPC C07K 16/2896; C07K 16/2809; C07K 2317/31; C07K 2317/52

(57) ABSTRACT

Disclosed are bispecific single-chain constructs, pharmaceutical compositions comprising the constructs, methods of treatment using the pharmaceutical compositions, polynucleotides encoding the constructs, and methods of making the constructs.

### CROSS-REFERENCE TO RELATED APPLICATIONS

This application claims the benefit of U.S. Application No. 63/476,766 filed on Dec. 22, 2022, the entire content of which is incorporated herein by reference.

### STATEMENT REGARDING FEDERALLY SPONSORED RESEARCH OR DEVELOPMENT

Not applicable.

### SEQUENCE LISTING

A Sequence Listing accompanies this application and is submitted as an XML file of the sequence listing named “650053_01038.xml” which is 30,846 bytes in size and was created on Dec. 21, 2023. The sequence listing is electronically submitted via Patent Center with the application and is incorporated herein by reference in its entirety.

### BACKGROUND

CD138, also known as Syndecan-1, is a transmembrane protein involved in cell adhesion. Although CD138 can be expressed in various cell types throughout the body, CD138 is highly expressed in both normal and malignant plasma cells. This has led to CD138 being one of the primary identifying biomarkers for multiple myeloma, although CD138 has been associated with many other cancer types as well.

### SUMMARY

In an aspect of the current disclosure, bispecific single-chain polypeptide constructs are provided. In some embodiments, the constructs comprise an anti-CD138 domain and an anti-CD3 domain. In some embodiments, the anti-CD138 domain comprises a first heavy chain and a first light chain and the anti-CD3 domain comprises a second heavy chain and a second light chain. In some embodiments, the anti-CD138 domain is SEQ ID NO: 1. In some embodiments, the anti-CD3 domain is SEQ ID NO: 2. In some embodiments, the first heavy chain comprises SEQ ID NO: 3. In some embodiments, the first light chain comprises SEQ ID NO: 4. In some embodiments, the second heavy chain comprises SEQ ID NO: 5. In some embodiments, the second light chain comprises SEQ ID NO: 6. In some embodiments, the constructs further comprise a linker. In some embodiments, the linker is a flexible hydrophobic linker. In some embodiments, the linker does not comprise a hinge region. In some embodiments, the linker comprises SEQ ID NO: 7. In some embodiments, the constructs further comprise an antibody Fc domain. In some embodiments, the Fc domain is an IgG Fc domain. In some embodiments, the Fc domain is IgG2 Fc domain. In some embodiments, the Fc domain comprises SEQ ID NO: 8. In some embodiments, the construct comprises, from N to C terminus, an anti-CD138 domain, a linker, an antibody Fc domain, a linker, an anti-CD3 domain. In some embodiments, the construct comprises, from N to C terminus, an anti-CD3 domain, a linker, an antibody Fc domain, a linker, an anti-CD138 domain. In some embodiments, the construct comprises SEQ ID NO: 9.

In another aspect of the current disclosure, pharmaceutical compositions are provided. In some embodiments, the pharmaceutical compositions comprise a construct comprising an anti-CD138 domain and an anti-CD3 domain; and a pharmaceutically acceptable carrier or excipient. In some embodiments, the anti-CD138 domain comprises a first heavy chain and a first light chain and the anti-CD3 domain comprises a second heavy chain and a second light chain. In some embodiments, the anti-CD138 domain is SEQ ID NO: 1. In some embodiments, the anti-CD3 domain is SEQ ID NO: 2. In some embodiments, the first heavy chain comprises SEQ ID NO: 3. In some embodiments, the first light chain comprises SEQ ID NO: 4. In some embodiments, the second heavy chain comprises SEQ ID NO: 5. In some embodiments, the second light chain comprises SEQ ID NO: 6. In some embodiments, the constructs further comprise a linker. In some embodiments, the linker is a flexible hydrophobic linker. In some embodiments, the linker does not comprise a hinge region. In some embodiments, the linker comprises SEQ ID NO: 7. In some embodiments, the constructs further comprise an antibody Fc domain. In some embodiments, the Fc domain is an IgG Fc domain. In some embodiments, the Fc domain is IgG2 Fc domain. In some embodiments, the Fc domain comprises SEQ ID NO: 8. In some embodiments, the construct comprises, from N to C terminus, an anti-CD138 domain, a linker, an antibody Fc domain, a linker, an anti-CD3 domain. In some embodiments, the construct comprises, from N to C terminus, an anti-CD3 domain, a linker, an antibody Fc domain, a linker, an anti-CD138 domain. In some embodiments, the construct comprises SEQ ID NO: 9.

In another aspect of the current disclosure, methods of treating a disease or disorder associated with CD138 expression are provided. In some embodiments, the methods comprise administering an effective amount of a pharmaceutical composition comprising a construct comprising an anti-CD138 domain and an anti-CD3 domain; and a pharmaceutically acceptable carrier or excipient to a subject in need thereof to treat the disease or disorder. In some embodiments, the disease or disorder is a cell-proliferative disease or disorder. In some embodiments, the cell proliferative disease or disorder is cancer. In some embodiments, the disease or disorder is multiple myeloma. In some embodiments, administering comprises contacting cells ex vivo with the pharmaceutical composition. In some embodiments, the method further comprises administering the cells to the subject after contacting. In some embodiments, the cells are T cells. In some embodiments, the cells are CD4+ T cells. In some embodiments, the cells are CD8⁺ T cells. In some embodiments, the cells are derived from the subject.

In another aspect of the current disclosure, polynucleotides are provided. In some embodiments, the polynucleotides encode a construct comprising an anti-CD138 domain and an anti-CD3 domain. In some embodiments, the anti-CD138 domain comprises a first heavy chain and a first light chain and the anti-CD3 domain comprises a second heavy chain and a second light chain. In some embodiments, the anti-CD138 domain is SEQ ID NO: 1. In some embodiments, the anti-CD3 domain is SEQ ID NO: 2. In some embodiments, the first heavy chain comprises SEQ ID NO: 3. In some embodiments, the first light chain comprises SEQ ID NO: 4. In some embodiments, the second heavy chain comprises SEQ ID NO: 5. In some embodiments, the second light chain comprises SEQ ID NO: 6. In some embodiments, the constructs further comprise a linker. In some embodiments, the linker is a flexible hydrophobic linker. In some embodiments, the linker does not comprise a hinge region. In some embodiments, the linker comprises SEQ ID NO: 7. In some embodiments, the constructs further comprise an antibody Fc domain. In some embodiments, the Fc domain is an IgG Fc domain. In some embodiments, the Fc domain is IgG2 Fc domain. In some embodiments, the Fc domain comprises SEQ ID NO: 8. In some embodiments, the construct comprises, from N to C terminus, an anti-CD138 domain, a linker, an antibody Fc domain, a linker, an anti-CD3 domain. In some embodiments, the construct comprises, from N to C terminus, an anti-CD3 domain, a linker, an antibody Fc domain, a linker, an anti-CD138 domain. In some embodiments, the construct comprises SEQ ID NO: 9.

In another aspect of the current disclosure, cells are provided. In some embodiments, the cells comprise a polynucleotide encoding a construct comprising an anti-CD138 domain and an anti-CD3 domain. In some embodiments, the anti-CD138 domain comprises a first heavy chain and a first light chain and the anti-CD3 domain comprises a second heavy chain and a second light chain. In some embodiments, the anti-CD138 domain is SEQ ID NO: 1. In some embodiments, the anti-CD3 domain is SEQ ID NO: 2. In some embodiments, the first heavy chain comprises SEQ ID NO: 3. In some embodiments, the first light chain comprises SEQ ID NO: 4. In some embodiments, the second heavy chain comprises SEQ ID NO: 5. In some embodiments, the second light chain comprises SEQ ID NO: 6. In some embodiments, the constructs further comprise a linker. In some embodiments, the linker is a flexible hydrophobic linker. In some embodiments, the linker does not comprise a hinge region. In some embodiments, the linker comprises SEQ ID NO: 7. In some embodiments, the constructs further comprise an antibody Fc domain. In some embodiments, the Fc domain is an IgG Fc domain. In some embodiments, the Fc domain is IgG2 Fc domain. In some embodiments, the Fc domain comprises SEQ ID NO: 8. In some embodiments, the construct comprises, from N to C terminus, an anti-CD138 domain, a linker, an antibody Fc domain, a linker, an anti-CD3 domain. In some embodiments, the construct comprises, from N to C terminus, an anti-CD3 domain, a linker, an antibody Fc domain, a linker, an anti-CD138 domain. In some embodiments, the construct comprises SEQ ID NO: 9.

In another aspect of the current disclosure, further cells are provided. In some embodiments, the cells comprise a construct comprising an anti-CD138 domain and an anti-CD3 domain. In some embodiments, the anti-CD138 domain comprises a first heavy chain and a first light chain and the anti-CD3 domain comprises a second heavy chain and a second light chain. In some embodiments, the anti-CD138 domain is SEQ ID NO: 1. In some embodiments, the anti-CD3 domain is SEQ ID NO: 2. In some embodiments, the first heavy chain comprises SEQ ID NO: 3. In some embodiments, the first light chain comprises SEQ ID NO: 4. In some embodiments, the second heavy chain comprises SEQ ID NO: 5. In some embodiments, the second light chain comprises SEQ ID NO: 6. In some embodiments, the constructs further comprise a linker. In some embodiments, the linker is a flexible hydrophobic linker. In some embodiments, the linker does not comprise a hinge region. In some embodiments, the linker comprises SEQ ID NO: 7. In some embodiments, the constructs further comprise an antibody Fc domain. In some embodiments, the Fc domain is an IgG Fc domain. In some embodiments, the Fc domain is IgG2 Fc domain. In some embodiments, the Fc domain comprises SEQ ID NO: 8. In some embodiments, the construct comprises, from N to C terminus, an anti-CD138 domain, a linker, an antibody Fc domain, a linker, an anti-CD3 domain. In some embodiments, the construct comprises, from N to C terminus, an anti-CD3 domain, a linker, an antibody Fc domain, a linker, an anti-CD138 domain. In some embodiments, the construct comprises SEQ ID NO: 9. In some embodiments, the cell is selected from a bacterial cell, an archaeal cell, a fungal cell, or an animal cell. In some embodiments, the cell is a human cell. In some embodiments, the human cell is a T cell.

### BRIEF DESCRIPTION OF THE FIGURES

FIG. 1 shows an exemplary schematic of the disclosed bispecific single-chain constructs. V_{H} is variable heavy chain, V_{L} is variable light chain, CH2 and CH3 are constant heavy chain domains 2 and 3 respectively.

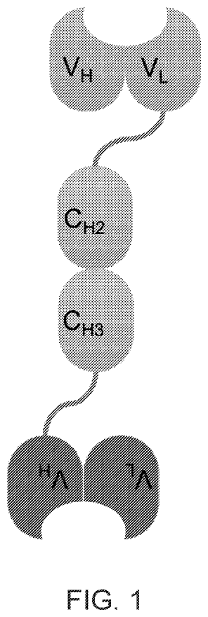

FIGS. 2A, 2B, 2C, 2D, 2E, 2F, 2G, and 2H show flow cytometry data demonstrating co-localization of T cells and target CD138⁺ cells after T cells were armed with the disclosed bispecific single-chain constructs. On the X axis is CELLTRACE violet, which corresponds to labelled T cells and on the y-axis is CELLTRACE yellow, which corresponds to labelled target cells expressing CD138.

FIG. 3 shows an exemplary sequence of one embodiment of the disclosed bispecific single-chain constructs.

FIGS. 4A, 4B, and 4C shows that the disclosed constructs effectively bind to both CD138 on target cells and CD3 on T cells and induce production of effector cytokines GM-CSF (FIG. 4A), IFNγ (FIG. 4B), and IL-10 (FIG. 4C) in media harvested after 24 hrs of co-culture of T cells and control (OCI AML2, which are CD138⁻) or experimental cells (OCI AML2 LV CD138, which are engineered to be CD138⁺ or K562 LV CD138, which are engineered to be CD138⁺) at a ratio 2:1, effector to target. OCI AML2 does not express CD138 but was engineered to express CD138 by the inventors (OCI AML2 LV CD138). Significance is measured by multiple T tests, *** p>0.001. Data expressed as mean=SD, n=3.

### DETAILED DESCRIPTION

### Bispecific Single-Chain Constructs

The inventors have generated a novel single-chain antibody from a human-derived antibody phage display library. This novel single-chain antibody binds to CD138. The sequence for the novel anti-CD138 domain was assembled as an amino acid sequence from a human derived phage display library. When back translating to DNA, the inventors optimized for a specific species, for certain GC ratios, and depleting uridine occurrence when DNA is translated to mRNA for protein synthesis. In addition, the nucleotide sequence for the anti-CD138 domain was optimized for expression in human cells. The inventors have incorporated this anti-CD138 single-chain antibody sequence into the disclosed bispecific single-chain constructs. This bispecific construct, also referred to as “CD138 Grappler,” recognizes both CD138 and CD3 on T cells and is expressed as a single protein chain. This eliminates post-expression modifications to the antibody such as the chemical conjugation required to fuse two IgG antibodies together that many other bispecific antibody constructs require. Thus, the disclosed CD138-CD3 bispecific single-chain construct possesses inherent advantages over bispecific antibody constructs that are chemically conjugated.

CD138 is associated with several malignancies including multiple myeloma. One strategy to target CD138 expressing cells, e.g., CD138 expressing cancer cells, is to design a bispecific construct that will simultaneously bind to CD138 expressing cells and bind to and activate T cells. Thus, the inventors have developed the disclosed novel bi-specific single-chain constructs that binds CD3 on T cells and CD138 on target cells, e.g., tumor cells. In some embodiments, the disclosed constructs are designed to cause the association of T cells with CD138 expressing cells and induce activation of the T cells. The inventors believe that this strategy will improve T cell targeting of CD138⁺ cells.

In some embodiments, disclosed constructs comprise scFvs to target CD3, e.g., SEQ ID NO: 2 and CD138, e.g., SEQ ID NO: 1, an IgG2 Fc region, e.g., SEQ ID NO: 8, and flexible, hydrophilic linkers connecting the scFvs to the Fc region of the antibody, e.g., SEQ ID NO: 7. Rather than using a traditional (GGGS)ₙ linker, the disclosed compounds comprise, in some embodiments, a flexible, hydrophilic linker, which is designed to improve the bioavailability and decrease the volume of distribution of the Grapplers, while still allowing for the flexibility traditional (GGGS)ₙ linkers impart. In some embodiments, the constructs comprise an Fc region which, in some embodiments, is derived from an IgG2 antibody. This subclass of IgG is designed to result in lower systemic responses to the Grappler. In addition, an Fc region is designed to allow the Grappler to use the FcRn recycling pathway to increase the half-life of the Grappler protein beyond what would be expected for a protein of its size.

Accordingly, in an aspect of the current disclosure, bispecific single-chain constructs are provided. In some embodiments, the constructs comprise an anti-CD138 domain and an anti-CD3 domain. In some embodiments, the anti-CD138 domain and the anti-CD3 domain are antibody single chain variable fragments (scFvs) which are made up of an antibody heavy chain and light chain linked together. Thus, in some embodiments, the anti-CD138 domain comprises a first heavy chain and a first light chain, and the anti-CD3 domain comprises a second heavy chain and a second light chain. In some embodiments, the first heavy chain (anti-CD138) comprises SEQ ID NO: 3. In some embodiments, the first light chain (anti-CD138) comprises SEQ ID NO: 4. In some embodiments, the second heavy chain comprises SEQ ID NO: 5. In some embodiments, the second light chain (anti-CD3) comprises SEQ ID NO: 6. In some embodiments, the anti-CD138 domain has the sequence SEQ ID NO: 1, or a sequence with at least about 50% identity, at least about 55% identity, at least about 60% identity, at least about 65% identity, at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to SEQ ID NO: 1. In some embodiments, the anti-CD3 domain is SEQ ID NO: 2, or a sequence with at least about 50% identity, at least about 55% identity, at least about 60% identity, at least about 65% identity, at least about 70% identity, at least about 75% identity, at least about 80% identity, at least about at least about 93% identity, at least about 94% identity, at least about 95% identity, at least about 96% identity, at least about 97% identity, at least about 98% identity, or at least about 99% identity to SEQ ID NO: 2. SEQ ID NO: 2 is derived from a humanized anti-CD38 monoclonal antibody (OKT3), see, e.g., U.S. Pat. No. 6,491,916.

In some embodiments, the constructs comprise a linker. The inventors believe that a flexible hydrophobic linker, which does not comprise a hinge region may be beneficial for properties of the disclosed constructs. Therefore, in some embodiments, the linker is a flexible hydrophobic linker which, in some embodiments, does not comprise a hinge region, e.g., the linker with the amino acid sequence SEQ ID NO: 7.

The inventors believe that the addition of an antibody Fc domain also improves the properties of the disclosed constructs by, for example, increasing the half-life of the construct in a subject. Accordingly, in some embodiments, the disclosed constructs comprise an antibody Fc domain. In some embodiments, the Fc domain is an IgG Fc domain. In some embodiments, the Fc domain is IgG2 Fc domain. In some embodiments, the Fc domain comprises SEQ ID NO: 8, or a sequence with at least about 90% identity to SEQ ID NO: 8. In some embodiments the Fc domain is SEQ ID NO: 8.

In some embodiments, the directionality of the construct may be defined. Accordingly, in some embodiments, the construct comprises, from N to C terminus, an anti-CD138 domain, a linker, an antibody Fc domain, a linker, an anti-CD3 domain. In other embodiments, the construct comprises, from N to C terminus, an anti-CD3 domain, a linker, an antibody Fc domain, a linker, an anti-CD138 domain. In some embodiments, the construct comprises SEQ ID NO: 9.

### Polynucleotides

Also contemplated in the current disclosure are polynucleotides that encode the disclosed constructs. Accordingly, in another aspect of the current disclosure, polynucleotides are provided. In some embodiments, the polynucleotides encode a bispecific single-chain construct comprising an anti-CD138 domain and an anti-CD3 domain. Thus, in some embodiments, the first heavy chain is encoded by a sequence comprising SEQ ID NO: 13. One of skill in the art would understand that certain modifications to the DNA sequence encoding the disclosed constructs, or portions thereof, could be modified to generate constructs with essentially the same amino acid structure, for example, through codon optimization or conservative codon usage. Thus, in some embodiments, the first heavy chain (anti-CD138) may be encoded by a sequence with at least about 50% identity, at least about 55% identity, at least about 60% identity, at least about 65% identity, at least about 70% identity, at least about 75% identity, at least about 80% identity, at least about at least about 93% identity, at least about 94% identity, at least about 95% identity, at least about 96% identity, at least about 97% identity, at least about 98% identity, or at least about 99% identity to SEQ ID NO: 13. In some embodiments, the first light chain (anti-CD138) is encoded by a sequence comprising SEQ ID NO: 15, or a sequence with at least about 50% identity, at least about 55% identity, at least about 60% identity, at least about 65% identity, at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to SEQ ID NO: 15. In some embodiments, the second heavy chain (anti-CD3) is encoded by a sequence comprising SEQ ID NO: 21, or a sequence with at least about 50% identity, at least about 55% identity, at least about 60% identity, at least about 65% identity, at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to SEQ ID NO: 21. In some embodiments, the second light chain (anti-CD3) is encoded by a sequence comprising SEQ ID NO: 23, or a sequence with at least about 50% identity, at least about 55% identity, at least about 60% identity, at least about 65% identity, at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to SEQ ID NO: 23. In some embodiments, the construct is encoded by a sequence comprising SEQ ID NO: 10, or a sequence with at least about 50% identity, at least about 55% identity, at least about 60% identity, at least about 65% identity, at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to SEQ ID NO: 10.

### Pharmaceutical Compositions

The disclosed bispecific constructs are contemplated to be used as a pharmaceutical intervention. Thus, in another aspect of the current disclosure, pharmaceutical compositions are provided. In some embodiments, the pharmaceutical compositions comprise a bispecific single-chain construct comprising an anti-CD138 domain and an anti-CD3 domain. As will be understood by one of skill in the art, the disclosed pharmaceutical compositions may be formulated such that they include appropriate pharmaceutical carriers or excipients to maximize the desired pharmaceutical composition's function. Such pharmaceutical formulations are considered routine.

In some embodiments, the pharmaceutical compositions are formulated for parenteral administration, i.e., they may be formulated for administration by subcutaneous (SC/SQ), intraperitoneal (IP), intravenous (IV), intradermal (ID), and intramuscular (IM) route. Suitably, the pharmaceutical compositions are formulated for intravenous administration. In some embodiments, the pharmaceutical compositions are formulated such that they consist only of GMP approved compounds, i.e., they have defined characteristics, and may be used in conjunction with GMP prepared cells, e.g., the compositions may be used in conjunction with cells intended for adoptive transfer therapy.

### Methods of Treatment

In another aspect of the current disclosure, methods of treating a disease or disorder associated with CD138 expression are provided. In some embodiments, the methods comprise administering an effective amount of a pharmaceutical composition comprising a bispecific single-chain construct comprising an anti-CD138 domain and an anti-CD3 domain to a subject in need thereof to treat the disease or disorder. As discussed above, CD138 is expressed on a variety of transformed cells, e.g., transformed cells present in a subject with a cell-proliferative disease or disorder. In some embodiments, the cell proliferative disease or disorder is multiple myeloma; however, it is to be understood that the diseases and disorders associated with CD138 expression are not limited to the foregoing examples. Accordingly, cancers that are characterized by CD138 expression on the surface of cancerous cells are also contemplated to be targets for treatment by administration of the disclosed compositions, pharmaceutical compositions and by use of the disclosed methods.

As used herein, “administration” may comprise any acceptable administrative route, e.g., intravenous administration or intrathecal administration. Alternatively, administration may comprise administration by percutaneous, intramuscular, intranasal, buccal, intrathecal, intracerebral, or intrarectal routes. The route of administration may be varied in any way, limited by the physical properties of the compounds being employed and the convenience of the subject and the caregiver, etc.

In some embodiments, the disclosed compositions, e.g., bispecific antibodies, can be directly administered to a subject in need thereof (e.g., a subject diagnosed with or suffering from a CD-138 disease or condition, such as multiple myeloma). By way of example, but not by way of limitation, administration of the bispecific antibody may include intravenous, intrathecal, intracranial, or intratumoral administration.

The inventors envision that in the disclosed methods, cells, e.g., T cells, may be isolated from the subject by leukapheresis, e.g., a subject with multiple myeloma, and contacted with the disclosed bispecific single-chain constructs or pharmaceutical compositions comprising the same, ex vivo. Thus, the cells, e.g., T cells, are pre-loaded with the construct. Without wishing to be limited by any theory or mechanism, the inventors believe that this strategy increases positive interactions between immune cells, e.g., T cells, and the disclosed constructs as compared to administration to a subject, or administration to a subject after infusion of T cells. In other words, the disclosed constructs are more likely to bind to the T cells ex vivo due to higher relative concentration of the construct to the T cells in comparison with administration of the disclosed constructs to the subject, e.g., intravenously, which requires that the disclosed constructs bind to the immune cells, e.g., T cells, and target cells in situ, i.e., in the context of the bloodstream, lymphatic system, or in tissues. In some embodiments, the T cells are stimulated ex vivo with, e.g., anti-CD3 antibodies and/or anti-CD28 antibodies and expanded by culturing the cells with interleukin 2 (IL-2). Then, the inventors envision that, in one embodiment, the cells are re-infused into the subject. Accordingly, the pre-loaded cells are brought into close contact with target cells, i.e., cells expressing CD138. Thus, the inventors believe that the immune cells, e.g., T cells, become stimulated in close proximity to the target cells and initiate a cytotoxic response and/or a cytokine-based inflammatory response targeting the CD138⁺ cells. The inventors envision that the immune cells may be, e.g., CD8⁺ T cells, CD4⁺ T cells, or a combination of CD8⁺ T cells and CD4⁺ T cells.

Thus, in one embodiment, T cells from a patient with CD138 positive cancerous tissue will be harvested via blood collection. Then the T cells will be armed with the CD138 Grappler. The armed T cells will be infused back into the patient. When the armed T cells come near CD138 positive cells, the CD138 Grappler would bind to the CD138 proteins on the cancerous cells. The T cells would then be able to release both lytic granzymes and inflammatory cytokines. The granzymes would induce lysis of the cancerous bound cell. The cytokines would be recruit other immune cells to the site of the cancerous tissue allowing for greater levels of targeted cell lysis.

### Cells

The instant disclosure provides polynucleotides that encode the disclosed novel bispecific single-chain constructs. Accordingly, in another aspect of the current disclosure, cells comprising the disclosed polynucleotides encoding the bispecific single-chain constructs are provided. In some embodiments, the cells are a mammalian cells, e.g., human cells. In some embodiments, the cells are HEK293 cells.

In addition to the cells provided by the instant disclosure, methods of making the disclosed bispecific single-chain constructs are provided. In some embodiments, the methods comprise introducing the disclosed polynucleotides into a cell and allowing the cell to express the polynucleotides to generate the disclosed bispecific single-chain constructs in vitro. In some embodiments, the constructs are further purified from the cells by means known in the art. In some embodiments, the constructs comprise affinity tags, e.g., streptavidin tags, histidine tags, FLAG tags, HA tags, etc., which allow their efficient purification.

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Illustrative Embodiments

1. A bispecific single-chain polypeptide construct comprising an anti-CD138 domain and an anti-CD3 domain.

2. The construct of embodiment 1, wherein the anti-CD138 domain comprises a first heavy chain and a first light chain and the anti-CD3 domain comprises a second heavy chain and a second light chain.

3. The construct of any one of embodiments 1 or 2, wherein the anti-CD138 domain is SEQ ID NO: 1.

4. The construct of any one of embodiments 1-3, wherein the anti-CD3 domain is SEQ ID NO: 2.

5. The construct of any one of embodiments 2-4, wherein the first heavy chain comprises SEQ ID NO: 3.

6. The construct of any one of embodiments 2-5, wherein the first light chain comprises SEQ ID NO: 4.

7. The construct of any one of embodiments 2-6, wherein the second heavy chain comprises SEQ ID NO: 5.

8. The construct of any one of embodiments 2-7, wherein the second light chain comprises SEQ ID NO: 6.

9. The construct of any one of embodiments 1-8, comprising a linker.

10. The construct of embodiment 9, wherein the linker is a flexible hydrophobic linker.

11. The construct of any one of embodiments 9 or 10, wherein the linker does not comprise a hinge region.

12. The construct of any one of embodiments 9-11, wherein the linker comprises SEQ ID NO: 7.

13. The construct of any one of embodiments 1-12, comprising an antibody Fc domain.

14. The construct of embodiment 13, wherein the Fc domain is an IgG Fc domain.

15. The construct of embodiments 13 or 14, wherein the Fc domain is IgG2 Fc domain.

16. The construct of any one of embodiments 13-15, wherein the Fc domain comprises SEQ ID NO: 8.

17. The construct of any one of embodiments 1-16, wherein the construct comprises, from N to C terminus, an anti-CD138 domain, a linker, an antibody Fc domain, a linker, an anti-CD3 domain.

18. The construct of any one of embodiments 1-16, wherein the construct comprises, from N to C terminus, an anti-CD3 domain, a linker, an antibody Fc domain, a linker, an anti-CD138 domain.

19. The construct of any one of embodiments 1-18, wherein the construct comprises SEQ ID NO: 9.

20. A pharmaceutical composition comprising the construct of any one of embodiments 1-19 and a pharmaceutically acceptable carrier or excipient.

21. A method of treating a disease or disorder associated with CD138 expression comprising administering an effective amount of the pharmaceutical composition of embodiment 19 to a subject in need thereof to treat the disease or disorder.

22. The method of embodiment 21, wherein the disease or disorder is a cell-proliferative disease or disorder.

23. The method of one of embodiment 22, wherein the cell proliferative disease or disorder is cancer.

24. The method of any one of embodiments 21-23, wherein the disease or disorder is multiple myeloma.

25. The method of any one of embodiments 21-24, wherein administering comprises contacting cells ex vivo with the pharmaceutical composition.

26. The method of embodiment 25, wherein the method further comprises administering the cells to the subject after contacting.

27. The method of any one of embodiment 25 or 26, wherein the cells are T cells.

28. The method of embodiment 27, wherein the cells are CD4⁺ T cells.

29. The method of embodiment 27, wherein the cells are CD8⁺ T cells.

30. The method of any one of embodiments 25-29, wherein the cells are derived from the subject.

31. A polynucleotide encoding the construct of any one of embodiments 1-19.

32. A cell comprising the polynucleotide of embodiment 31.

33. A cell comprising the construct of any one of embodiments 1-19.

34. The cell of embodiment 32 or 33, wherein the cell is selected from a bacterial cell, an archaeal cell, a fungal cell, or an animal cell.

35. The cell of embodiment 34, wherein the cell is a human cell.

36. The cell of embodiment 35, wherein the human cell is a T cell.

### EXAMPLES

The following Examples are illustrative and should not be interpreted to limit the scope of the claimed subject matter.

### Example 1—Generation and Testing of Bispecific Single-Chain Constructs

The sequences encoding the disclosed bispecific single-chain constructs were designed by the inventors and placed into a pcDNA 3.4 TOPO expression vector by a vendor. The Grappler plasmids were cloned into XL-1 gold E. coli and harvested plasmids via giga preps. Plasmids were transfected into Expi293 cells (HEK293 cells). Transfected cells were given 7 days to express the Grapplers into the supernatants. The Grapplers comprised antibody Fc domains and, therefore, were purified from the supernatants with protein A beads. BCA assays were used to determine protein yields.

The inventors tested the function of the disclosed bispecific single-chain constructs by performing an assay designed to test whether the constructs bound to both T cells and target cells.

Primary T cells were isolated from whole blood acquired from Stemcell (Vancouver, BC, #70507.6). Primary T cell cultures may be expanded and incubated with the anti-CD3/anti-CD138 bispecific single-chain constructs for 1 hr before aliquots were placed in cryo storage. To eliminate the need for excessive isotype controls, CELLTRACE dyes were used instead of fluorescent-tagged antibodies. Briefly, T cells armed with the bispecific single-chain constructs were thawed and counted. One million cells of both T cells and target cells were dyed per reaction. Armed T cells were dyed with CELLTRACE violet (#C34571) and target cells were dyed with CELLTRACE yellow (#C34573). Cells were incubated in the dark with their respective dye diluted 1:10,000 in 1×PBS for 10 min, inverting tubes every 2 minutes. Dyed cells were incubated with heat inactivated FBS for 1 min. Target cells and armed T cells were incubated together at a 1:1 T cell: target cell ratio at 21° C. for 30 min in the dark while shaking. Cells were washed and fixed with 1% paraformaldehyde (#15710, Electron Microscopy Sciences). Dyed cells were run LSRII (BD) flow cytometer and analyzed with Flowjo v 10.

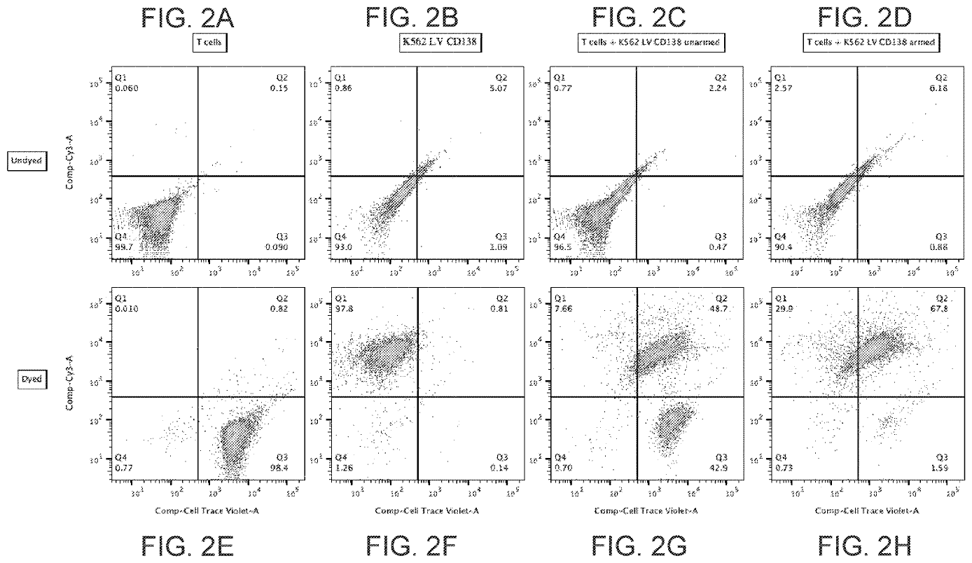

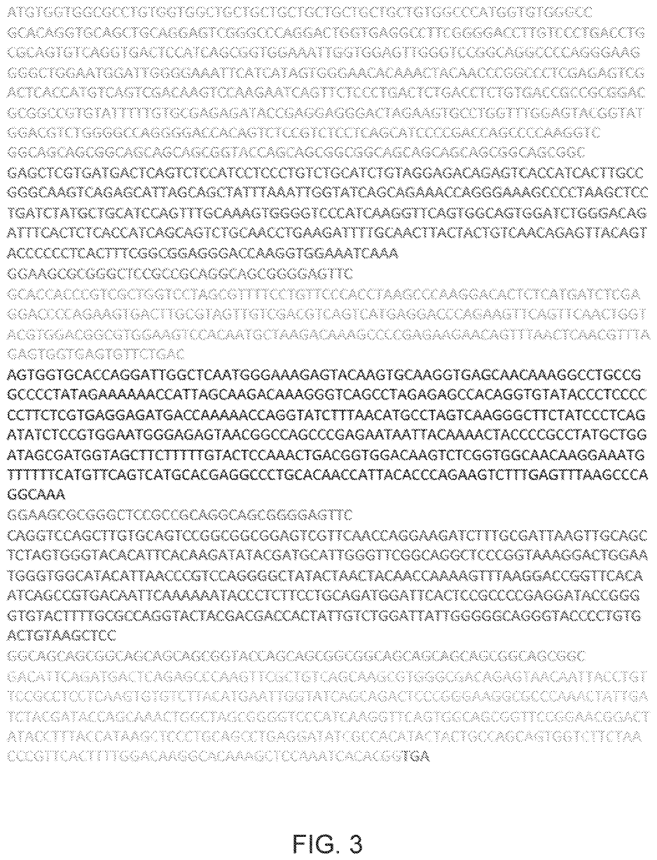

T cells pre-armed with the Grapplers were dyed with the CELLTRACE violet when applicable. Target cells (cells expressing CD138) were dyed with CELLTRACE yellow when applicable. The first two panels (FIGS. 2A and 3B) show undyed T cells and target cells. Moving to the right, the next panels (FIGS. 2C and 3D) show two distinct populations when the two cell populations are incubated without the respective Grappler (bispecific single-chain construct). When Grappler is added, the two different cell lines appear as one population, as can be observed in FIG. 2D. This provides initial evidence that the Grappler is bringing the two cell populations together. In the bottom row, cells were dyed with their respective CELLTRACE dye. The first two panels (FIGS. 2E and 2F) show that when T cells or target cells are dyed, they show up as being dyed. In the next panel (FIG. 2G), there is some co-localization occurring naturally without the Grapplers, likely due to the donor, from whom the T cells originated, having some previous immune response to a protein on the target cells. Lastly, in FIG. 2H, we show primarily a single population largely in the double positive quadrant. This indicates that when T cells armed with the Grappler are incubated with target cells expressing the protein of interest, nearly all the target cells become bound to the T cells armed with the respective Grappler. Taken together, these results show that the CD138 Grappler can bind to both T cells and cells expressing CD138.

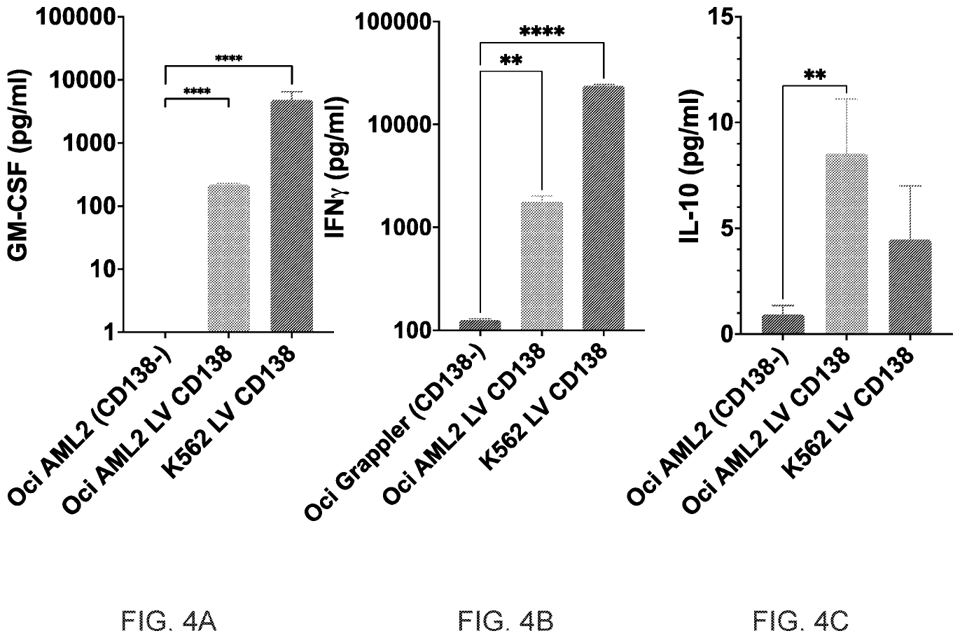

The inventors engineered the tumor cell line OCI AML2 to express CD138 (OCI AML2 LV CD138), incubated control cells (OCI AML2) and CD138⁺ experimental cells (OCI AML2 LV CD138 or K562 LV CD138) with T cells armed with the disclosed constructs at a 2:1 effector:target ratio (100 ng of construct per 1 million T cells). The inventors demonstrated that the disclosed constructs allow T cells to bind to target cells produce the inflammatory cytokines GM-CSF (FIG. 4A) and IFNγ (FIG. 4B), as well as the cytokine IL-10 (FIG. 4C). These data support the hypothesis that the disclosed constructs function to bring target and effector cells into close proximity and induce effector cells to produce inflammatory cytokines.

### Example 2—Treatment of Cancer with the Disclosed Bispecific Single-Chain Constructs

In one example, a subject suffering from cancer, e.g., multiple myeloma, is administered a therapeutically effective amount of a pharmaceutical composition comprising the disclosed bispecific single-chain constructs. The pharmaceutical composition may suitably be administered by any route that is indicated by the particular treatment needs of the subject, e.g., intravenously. Signs and symptoms of the cancer may be reduced by the administration of the pharmaceutical composition. Treatment may be administered daily, every other day, every third day, once a week, once a month, or on a schedule as determined by the patient's progress, pursuant to a physician's decision. It is anticipated that the subject may experience a reduction in signs or symptoms of the cancer as compared to an untreated subject. Methods of measuring reductions in signs and symptoms of cancer are known in the art, e.g., reduction in tumor burden, activation or differentiation of tumor-specific immune cells, e.g., tumor infiltrating lymphocytes (TILs).

### Example 3—Treatment of Cancer by Administration of Immune Cells Pre-Treated with the Disclosed Bispecific Single-Chain Constructs

In one example, a subject suffering from cancer, e.g., multiple myeloma, is administered a therapeutically effective amount of a pharmaceutical composition comprising cells that have been contacted, or “pre-treated” or “armed,” with the disclosed bispecific single-chain constructs in vitro or ex vivo. The cells may suitably be administered by any route that is indicated by the particular treatment needs of the subject, e.g., intravenously, intratumorally. The cells may be any suitable CD3-expressing cell, e.g., T cells. The cells may be autologous cells, e.g., autologous T cells. Signs and symptoms of the cancer may be reduced by the administration of the cells. Treatment may be administered daily, every other day, every third day, once a week, once a month, or on a schedule as determined by the patient's progress, pursuant to a physician's decision. It is anticipated that the subject may experience a reduction in signs or symptoms of the cancer as compared to an untreated subject. Methods of measuring reductions in signs and symptoms of cancer are known in the art, e.g., reduction in tumor burden, activation or differentiation of tumor-specific immune cells, e.g., tumor infiltrating lymphocytes (TILs).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

Sequences

| SEQ ID NO | Sequence | Description |
|---|---|---|
| 1 | AQVQLQESGPGLVRPSGTLSLTCAVSGDSISGGNWWSWVR | anti-CD138 |
| | QAPGKGLEWIGEIHHSGNTNYNPALESRLTMSVDKSKNQFS | scFv |
| | LTLTSVTAADAAVYFCARDTEEGLEVPGLEYGMDVWGQGT | |
| | TVSVSSASPTSPKVGSSGSSSGTSSGGSSSSGSGELVMTQSPS | |
| | SLSASVGDRVTITCRASQSISSYLNWYQQKPGKAPKLLIYAA | |
| | SSLQSGVPSRFSGSGSGTDFTLTISSLQPEDFATYYCQQSYST | |
| | PLTFGGGTKVEIK | |
| 2 | QVQLVQSGGGVVQPGRSLRLSCSSSGYTFTRYTMHWVRQA | anti-CD3 scFv |
| | PGKGLEWVAYINPSRGYTNYNQKFKDRFTISRDNSKNTLFL | |
| | QMDSLRPEDTGVYFCARYYDDHYCLDYWGQGTPVTVSSGS | |
| | SGSSSGTSSGGSSSSGSGDIQMTQSPSSLSASVGDRVTITCSA | |
| | SSSVSYMNWYQQTPGKAPKLLIYDTSKLASGVPSRFSGSGS | |
| | GTDYTFTISSLQPEDIATYYCQQWSSNPFTFGQGTKLQITR | |
| 3 | AQVQLQESGPGLVRPSGTLSLTCAVSGDSISGGNWWSWVR | anti-CD138 |
| | QAPGKGLEWIGEIHHSGNTNYNPALESRLTMSVDKSKNQFS | Heavy chain |
| | LTLTSVTAADAAVYFCARDTEEGLEVPGLEYGMDVWGQGT | |
| | TVSVSSASPTSPKV | |
| 4 | ELVMTQSPSSLSASVGDRVTITCRASQSISSYLNWYQQKPGK | anti-CD138 |
| | APKLLIYAASSLQSGVPSRFSGSGSGTDFTLTISSLQPEDFAT | Light chain |
| | YYCQQSYSTPLTFGGGTKVEIK | |
| 5 | QVQLVQSGGGVVQPGRSLRLSCSSSGYTFTRYTMHWVRQA | anti-CD3 |
| | PGKGLEWVAYINPSRGYTNYNQKFKDRFTISRDNSKNTLFL | Heavy chain |
| | QMDSLRPEDTGVYFCARYYDDHYCLDYWGQGTPVTVSS | |
| 6 | DIQMTQSPSSLSASVGDRVTITCSASSSVSYMNWYQQTPGK | anti-CD3 Light |
| | APKLLIYDTSKLASGVPSRFSGSGSGTDYTFTISSLQPEDIATY | chain |
| | YCQQWSSNPFTFGQGTKLQITR | |
| 7 | GSAGSAAGSGEF | Linker sequence |
| 8 | ASTKGPSVFPLAPCSRSTSESTAALGCLVKDYFPEPVTVSWN | Fc domain |
| | SGALTSGVHTFPAVLQSSGLYSLSSVVTVPSSNFGTQTYTCN | |
| | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDISVEWESNGQPENNYKTTPPMLDSDGSFFLYSKLT | |
| | VDKSRWQQGNVFSCSVMHEALHNHYTQKSLSLSPGK | |
| 9 | MWWRLWWLLLLLLLLWPMVWAQVQLQESGTELVKPGAS | Full sequence |
| | VKLSCKASGYTFTSYWMHWMKQRPGQGLEWIGNINPSNGG | of the construct |
| | TNYNEKFKNKATLTVDKSSSTAYMQLSSLTSEDSAVYYCAR | (AA) |
| | RDYYYGSSYGFDVWGTGTTVTVSSGGGGSGGGGSGGGGSD | |
| | IVMTQSPASQSASLGESVTITCLASQTIGTWLAWYQQKPGKS | |
| | PQFLIYAATSLADGVPSRFSGSGSGTKFSFKISSLQAEDFVSY | |
| | YCQQLYSTPFTFGGGTKLEIKGSAGSAAGSGEFAPPVAGPSV | |
| | FLFPPKPKDTLMISRTPEVTCVVVDVSHEDPEVQFNWYVDG | |
| | VEVHNAKTKPREEQFNSTFRVVSVLTVVHQDWLNGKEYKC | |
| | KVSNKGLPAPIEKTISKTKGQPREPQVYTLPPSREEMTKNQV | |
| | SLTCLVKGFYPSDISVEWESNGQPENNYKTTPPMLDSDGSFF | |
| | LYSKLTVDKSRWQQGNVFSCSVMHEALHNHYTQKSLSLSP | |
| | GKGSAGSAAGSGEFQVQLVQSGGGVVQPGRSLRLSCSSSGY | |
| | TFTRYTMHWVRQAPGKGLEWVAYINPSRGYTNYNQKFKD | |
| | RFTISRDNSKNTLFLQMDSLRPEDTGVYFCARYYDDHYCLD | |
| | YWGQGTPVTVSSGSSGSSSGTSSGGSSSSGSGDIQMTQSPSS | |
| | LSASVGDRVTITCSASSSVSYMNWYQQTPGKAPKLLIYDTS | |
| | KLASGVPSRFSGSGSGTDYTFTISSLQPEDIATYYCQQWSSNP | |
| | FTFGQGTKLQITR | |
| 10 | ATGTGGTGGCGACTTTGGTGGCTGCTGTTGCTGCTGCTTC | Full sequence |
| | TGCTGTGGCCTATGGTCTGGGCCCAAGTCCAGCTGCAAGA | of the construct |
| | GTCTGGCACAGAACTGGTTAAGCCAGGCGCCAGCGTGAA | (nucleotide) |
| | GCTGTCTTGTAAAGCCAGCGGCTACACCTTCACCAGCTAC | |
| | TGGATGCACTGGATGAAGCAGAGGCCTGGACAGGGACTC | |
| | GAGTGGATCGGCAACATCAATCCTAGCAACGGCGGCACC | |
| | AACTACAACGAGAAGTTCAAGAACAAGGCCACACTGACC | |
| | GTGGACAAGAGCAGCTCTACAGCCTACATGCAGCTGAGC | |
| | AGCCTGACAAGCGAGGACAGCGCCGTGTACTACTGCGCC | |
| | AGAAGAGACTACTACTACGGCAGCAGCTACGGCTTCGAT | |
| | GTGTGGGGAACTGGCACCACCGTGACAGTTTCTAGCGGA | |
| | GGCGGAGGAAGTGGTGGCGGAGGTTCTGGCGGCGGAGG | |
| | ATCTGATATCGTGATGACACAGAGCCCTGCCAGCCAGTCT | |
| | GCCTCTCTGGGAGAGTCTGTGACCATCACATGTCTGGCCA | |
| | GCCAGACCATTGGAACATGGCTGGCCTGGTATCAGCAGA | |
| | AACCTGGCAAGAGCCCTCAGTTCCTGATCTACGCCGCCAC | |
| | ATCTCTGGCTGATGGCGTGCCAAGCAGATTTTCTGGCTCT | |
| | GGCAGCGGCACAAAGTTCAGCTTCAAGATCAGCTCCCTG | |
| | CAGGCCGAGGACTTCGTGTCCTACTATTGCCAGCAGCTGT | |
| | ACTCTACCCCTTTCACCTTTGGCGGAGGCACCAAGCTGGA | |
| | AATCAAGGGCTCTGCTGGATCTGCCGCCGGATCTGGCGA | |
| | ATTTGCTCCTCCTGTTGCCGGACCTAGCGTGTTCCTGTTTC | |
| | CTCCAAAGCCTAAGGACACCCTGATGATCAGCAGGACCC | |
| | CTGAAGTGACCTGTGTGGTGGTGGATGTGTCCCACGAGG | |
| | ATCCCGAGGTGCAGTTCAATTGGTACGTGGACGGCGTGG | |
| | AAGTGCACAACGCCAAGACCAAGCCTAGAGAGGAACAGT | |
| | TCAACAGCACCTTCAGAGTGGTGTCCGTGCTGACAGTGGT | |
| | GCATCAGGACTGGCTGAACGGCAAAGAGTACAAGTGCAA | |
| | GGTGTCCAACAAGGGCCTGCCTGCTCCTATCGAGAAAAC | |
| | CATCAGCAAGACAAAGGGCCAGCCTCGCGAGCCTCAGGT | |
| | TTACACACTGCCTCCAAGCCGGGAAGAGATGACCAAGAA | |
| | TCAGGTGTCCCTGACCTGCCTGGTCAAGGGCTTCTACCCT | |
| | AGCGACATCAGCGTGGAATGGGAGAGCAATGGCCAGCCT | |
| | GAGAACAACTACAAGACCACACCTCCTATGCTGGACAGC | |
| | GACGGCTCATTCTTCCTGTACAGCAAGCTGACTGTGGATA | |
| | AGTCCCGGTGGCAGCAGGGCAATGTGTTCAGCTGTTCTGT | |
| | GATGCACGAGGCCCTGCACAACCACTACACCCAGAAGTC | |
| | TCTGTCTCTGAGCCCTGGCAAGGGAAGCGCAGGTTCTGCT | |
| | GCTGGAAGCGGAGAATTTCAGGTGCAGCTGGTTCAGTCA | |
| | GGCGGAGGTGTTGTGCAGCCTGGCAGAAGCCTGAGACTG | |
| | AGCTGTAGCAGCTCCGGATACACATTCACCCGGTACACC | |
| | ATGCACTGGGTTCGACAGGCCCCTGGCAAAGGACTGGAA | |
| | TGGGTCGCCTACATCAACCCCAGCCGGGGCTACACCAATT | |
| | ATAACCAGAAGTTTAAGGACCGGTTCACCATCAGCCGGG | |
| | ACAACAGCAAGAATACCCTGTTCCTGCAGATGGACAGCC | |
| | TGCGGCCTGAAGATACCGGCGTGTACTTTTGCGCCCGGTA | |
| | CTACGACGACCACTACTGCCTGGATTATTGGGGCCAGGG | |
| | CACACCTGTGACCGTGTCCAGTGGTAGCAGTGGCTCTAGC | |
| | TCTGGAACAAGCAGCGGCGGATCTTCTTCTAGCGGCTCCG | |
| | GCGATATCCAGATGACCCAGTCTCCAAGCTCTCTGAGCGC | |
| | CTCTGTGGGCGACAGAGTGACAATTACATGTAGCGCCAG | |
| | CTCCAGCGTGTCCTATATGAATTGGTATCAACAGACCCCA | |
| | GGCAAGGCCCCAAAGCTGCTGATCTATGATACCTCCAAG | |
| | CTGGCCTCCGGCGTGCCATCTAGATTTTCCGGAAGCGGCA | |
| | GCGGAACCGATTACACGTTCACCATTAGCAGCCTGCAGC | |
| | CAGAGGACATTGCCACCTATTACTGCCAGCAGTGGTCCA | |
| | GCAATCCCTTCACCTTCGGCCAGGGGACCAAGCTGCAGA | |
| | TCACCAGATAA | |
| 11 | MWWRLWWLLLLLLLLWPMVWA | HMM38 signal |
| | | sequence (AA) |
| 12 | ATGTGGTGGCGCCTGTGGTGGCTGCTGCTGCTGCTGCTGC | HMM38 signal |
| | TGCTGTGGCCCATGGTGTGGGCC | sequence |
| | | (Nucleotide) |
| 13 | GCACAGGTGCAGCTGCAGGAGTCGGGCCCAGGACTGGTG | anti-CD138 |
| | AGGCCTTCGGGGACCTTGTCCCTGACCTGCGCAGTGTCAG | Heavy chain |
| | GTGACTCCATCAGCGGTGGAAATTGGTGGAGTTGGGTCC | (nucleotide) |
| | GGCAGGCCCCAGGGAAGGGGCTGGAATGGATTGGGGAA | |
| | ATTCATCATAGTGGGAACACAAACTACAACCCGGCCCTC | |
| | GAGAGTCGACTCACCATGTCAGTCGACAAGTCCAAGAAT | |
| | CAGTTCTCCCTGACTCTGACCTCTGTGACCGCCGCGGACG | |
| | CGGCCGTGTATTTTTGTGCGAGAGATACCGAGGAGGGAC | |
| | TAGAAGTGCCTGGTTTGGAGTACGGTATGGACGTCTGGG | |
| | GCCAGGGGACCACAGTCTCCGTCTCCTCAGCATCCCCGAC | |
| | CAGCCCCAAGGTC | |
| 14 | GGCAGCAGCGGCAGCAGCAGCGGTACCAGCAGCGGCGG | CD138 linker |
| | CAGCAGCAGCAGCGGCAGCGGC | (nucleotide) |
| 15 | GAGCTCGTGATGACTCAGTCTCCATCCTCCCTGTCTGCAT | anti-CD138 |
| | CTGTAGGAGACAGAGTCACCATCACTTGCCGGGCAAGTC | Light chain |
| | AGAGCATTAGCAGCTATTTAAATTGGTATCAGCAGAAAC | (nucleotide) |
| | CAGGGAAAGCCCCTAAGCTCCTGATCTATGCTGCATCCAG | |
| | TTTGCAAAGTGGGGTCCCATCAAGGTTCAGTGGCAGTGG | |
| | ATCTGGGACAGATTTCACTCTCACCATCAGCAGTCTGCAA | |
| | CCTGAAGATTTTGCAACTTACTACTGTCAACAGAGTTACA | |
| | GTACCCCCCTCACTTTCGGCGGAGGGACCAAGGTGGAAA | |
| | TCAAA | |
| 16 | GGAAGCGCGGGCTCCGCCGCAGGCAGCGGGGAGTTC | Linker sequence |
| | | (nucleotide) |
| 17 | GCACCACCCGTCGCTGGTCCTAGCGTTTTCCTGTTCCCAC | Fc domain |
| | CTAAGCCCAAGGACACTCTCATGATCTCGAGGACCCCAG | (nuc) |
| | AAGTGACTTGCGTAGTTGTCGACGTCAGTCATGAGGACCC | |
| | AGAAGTTCAGTTCAACTGGTACGTGGACGGCGTGGAAGT | |
| | CCACAATGCTAAGACAAAGCCCCGAGAAGAACAGTTTAA | |
| | CTCAACGTTTAGAGTGGTGAGTGTTCTGACAGTGGTGCAC | |
| | CAGGATTGGCTCAATGGGAAAGAGTACAAGTGCAAGGTG | |
| | AGCAACAAAGGCCTGCCGGCCCCTATAGAAAAAACCATT | |
| | AGCAAGACAAAGGGTCAGCCTAGAGAGCCACAGGTGTAT | |
| | ACCCTCCCCCCTTCTCGTGAGGAGATGACCAAAAACCAG | |
| | GTATCTTTAACATGCCTAGTCAAGGGCTTCTATCCCTCAG | |
| | ATATCTCCGTGGAATGGGAGAGTAACGGCCAGCCCGAGA | |
| | ATAATTACAAAACTACCCCGCCTATGCTGGATAGCGATG | |
| | GTAGCTTCTTTTTGTACTCCAAACTGACGGTGGACAAGTC | |
| | TCGGTGGCAACAAGGAAATGTTTTTTCATGTTCAGTCATG | |
| | CACGAGGCCCTGCACAACCATTACACCCAGAAGTCTTTG | |
| | AGTTTAAGCCCAGGCAAA | |
| 18 | GCACCACCCGTCGCTGGTCCTAGCGTTTTCCTGTTCCCAC | CH2 |
| | CTAAGCCCAAGGACACTCTCATGATCTCGAGGACCCCAG | (nucleotide) |
| | AAGTGACTTGCGTAGTTGTCGACGTCAGTCATGAGGACCC | |
| | AGAAGTTCAGTTCAACTGGTACGTGGACGGCGTGGAAGT | |
| | CCACAATGCTAAGACAAAGCCCCGAGAAGAACAGTTTAA | |
| | CTCAACGTTTAGAGTGGTGAGTGTTCTGAC | |
| 19 | AGTGGTGCACCAGGATTGGCTCAATGGGAAAGAGTACAA | CH3 |
| | GTGCAAGGTGAGCAACAAAGGCCTGCCGGCCCCTATAGA | (Nucleotide) |
| | AAAAACCATTAGCAAGACAAAGGGTCAGCCTAGAGAGCC | |
| | ACAGGTGTATACCCTCCCCCCTTCTCGTGAGGAGATGACC | |
| | AAAAACCAGGTATCTTTAACATGCCTAGTCAAGGGCTTCT | |
| | ATCCCTCAGATATCTCCGTGGAATGGGAGAGTAACGGCC | |
| | AGCCCGAGAATAATTACAAAACTACCCCGCCTATGCTGG | |
| | ATAGCGATGGTAGCTTCTTTTTGTACTCCAAACTGACGGT | |
| | GGACAAGTCTCGGTGGCAACAAGGAAATGTTTTTTCATGT | |
| | TCAGTCATGCACGAGGCCCTGCACAACCATTACACCCAG | |
| | AAGTCTTTGAGTTTAAGCCCAGGCAAA | |
| 20 | GGAAGCGCGGGCTCCGCCGCAGGCAGCGGGGAGTTC | Linker sequence |
| | | (nucleotide) |
| 21 | CAGGTCCAGCTTGTGCAGTCCGGCGGCGGAGTCGTTCAA | anti-CD3 |
| | CCAGGAAGATCTTTGCGATTAAGTTGCAGCTCTAGTGGGT | Heavy chain |
| | ACACATTCACAAGATATACGATGCATTGGGTTCGGCAGG | (nucleotide) |
| | CTCCCGGTAAAGGACTGGAATGGGTGGCATACATTAACC | |
| | CGTCCAGGGGCTATACTAACTACAACCAAAAGTTTAAGG | |
| | ACCGGTTCACAATCAGCCGTGACAATTCAAAAAATACCC | |
| | TCTTCCTGCAGATGGATTCACTCCGCCCCGAGGATACCGG | |
| | GGTGTACTTTTGCGCCAGGTACTACGACGACCACTATTGT | |
| | CTGGATTATTGGGGGCAGGGTACCCCTGTGACTGTAAGCT | |
| | CC | |
| 22 | GGCAGCAGCGGCAGCAGCAGCGGTACCAGCAGCGGCGG | CD138 linker |
| | CAGCAGCAGCAGCGGCAGCGGC | (nucleotide) |
| 23 | GACATTCAGATGACTCAGAGCCCAAGTTCGCTGTCAGCA | anti-CD3 Light |
| | AGCGTGGGCGACAGAGTAACAATTACCTGTTCCGCCTCCT | chain |
| | CAAGTGTGTCTTACATGAATTGGTATCAGCAGACTCCCGG | (nucleotide) |
| | GAAGGCGCCCAAACTATTGATCTACGATACCAGCAAACT | |
| | GGCTAGCGGGGTCCCATCAAGGTTCAGTGGCAGCGGTTC | |
| | CGGAACGGACTATACCTTTACCATAAGCTCCCTGCAGCCT | |
| | GAGGATATCGCCACATACTACTGCCAGCAGTGGTCTTCTA | |
| | ACCCGTTCACTTTTGGACAAGGCACAAAGCTCCAAATCAC | |
| | ACGG | |
| 24 | GSSGSSSGTSSGGSSSSGSG | CD138 linker |
| | | (AA) |
| 25 | APPVAGPSVFLFPPKPKDTLMISRTPEVTCVVVDVSHEDPEV | CH2 (AA) |
| | QFNWYVDGVEVHNAKTKPREEQFNSTFRVVSVLTVVHQD | |
| | WLNGKEYKCKVSNKGLPAPIEKTISKTK | |
| 26 | GQPREPQVYTLPPSREEMTKNQVSLTCLVKGFYPSDISVEWE | CH3 (AA) |
| | SNGQPENNYKTTPPMLDSDGSFFLYSKLTVDKSRWQQGNV | |
| | FSCSVMHEALHNHYTQKSLSLSPGK | |

We claim:

1. A bispecific single-chain polypeptide construct comprising an anti-CD138 domain and an anti-CD3 domain, wherein the anti-CD138 domain comprises SEQ ID NO: 1 or a sequence with at least 85% identity to SEQ ID NO: 1.

2. The construct of claim 1, wherein the anti-CD138 domain comprises a first heavy chain and a first light chain and the anti-CD3 domain comprises a second heavy chain and a second light chain.

3. The construct of claim 1, wherein the anti-CD138 domain is SEQ ID NO: 1.

4. The construct of claim 1, wherein the anti-CD3 domain comprises SEQ ID NO: 2 or a sequence with at least 85% identity to SEQ ID NO: 2.

5. The construct of claim 2, wherein the first heavy chain comprises SEQ ID NO: 3.

6. The construct of claim 2, wherein the first light chain comprises SEQ ID NO: 4.

7. The construct of claim 2, wherein the second heavy chain comprises SEQ ID NO: 5.

8. The construct of claim 2, wherein the second light chain comprises SEQ ID NO: 6.

9. The construct of claim 1, comprising a linker.

10-12. (canceled)

13. The construct of claim 1, comprising an antibody Fc domain.

14. The construct of claim 13, wherein the Fc domain is an IgG Fc domain.

15-16. (canceled)

17. The construct of claim 1, wherein the construct comprises, from N to C terminus, an anti-CD138 domain, a linker, an antibody Fc domain, a linker, an anti-CD3 domain.

18. (canceled)

19. The construct of claim 1, wherein the construct comprises SEQ ID NO: 9.

20. A pharmaceutical composition comprising the construct of claim 1 and a pharmaceutically acceptable carrier or excipient.

21. A method of treating a disease or disorder associated with CD138 expression comprising administering an effective amount of the pharmaceutical composition of claim 20 to a subject in need thereof to treat the disease or disorder.

22. The method of claim 21, wherein the disease or disorder is a cell-proliferative disease or disorder.

23. The method of claim 22, wherein the cell proliferative disease or disorder is cancer.

24. The method of claim 22, wherein the disease or disorder is multiple myeloma.

25-30. (canceled)

31. A polynucleotide encoding the construct of claim 1.

32. A cell comprising the polynucleotide of claim 31.

33-36. (canceled)


SEQUENCE LISTING (26 sequences)

SEQ ID NO: 1 - protein, 262 aa, synthetic construct
AQVQLQESGPGLVRPSGTLSLTCAVSGDSISGGNWWSWVRQAPGKGLEWIGEIHHSGNTNYNPALESRLTMSVDKSKNQFSLTLTSVTAADAAVYFCARDTEEGLEVPGLEYGMDVWGQGTTVSVSSASPTSPKVGSSGSSSGTSSGGSSSSGSGELVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 2 - protein, 246 aa, synthetic construct
QVQLVQSGGGVVQPGRSLRLSCSSSGYTFTRYTMHWVRQAPGKGLEWVAYINPSRGYTNYNQKFKDRFTISRDNSKNTLFLQMDSLRPEDTGVYFCARYYDDHYCLDYWGQGTPVTVSSGSSGSSSGTSSGGSSSSGSGDIQMTQSPSSLSASVGDRVTITCSASSSVSYMNWYQQTPGKAPKLLIYDTSKLASGVPSRFSGSGSGTDYTFTISSLQPEDIATYYCQQWSSNPFTFGQGTKLQITR

SEQ ID NO: 3 - protein, 135 aa, synthetic construct
AQVQLQESGPGLVRPSGTLSLTCAVSGDSISGGNWWSWVRQAPGKGLEWIGEIHHSGNTNYNPALESRLTMSVDKSKNQFSLTLTSVTAADAAVYFCARDTEEGLEVPGLEYGMDVWGQGTTVSVSSASPTSPKV

SEQ ID NO: 4 - protein, 107 aa, synthetic construct
ELVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 5 - protein, 119 aa, synthetic construct
QVQLVQSGGGVVQPGRSLRLSCSSSGYTFTRYTMHWVRQAPGKGLEWVAYINPSRGYTNYNQKFKDRFTISRDNSKNTLFLQMDSLRPEDTGVYFCARYYDDHYCLDYWGQGTPVTVSS

SEQ ID NO: 6 - protein, 107 aa, synthetic construct
DIQMTQSPSSLSASVGDRVTITCSASSSVSYMNWYQQTPGKAPKLLIYDTSKLASGVPSRFSGSGSGTDYTFTISSLQPEDIATYYCQQWSSNPFTFGQGTKLQITR

SEQ ID NO: 7 - protein, 12 aa, synthetic construct
GSAGSAAGSGEF

SEQ ID NO: 8 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 9 - protein, 751 aa, synthetic construct
MWWRLWWLLLLLLLLWPMVWAQVQLQESGTELVKPGASVKLSCKASGYTFTSYWMHWMKQRPGQGLEWIGNINPSNGGTNYNEKFKNKATLTVDKSSSTAYMQLSSLTSEDSAVYYCARRDYYYGSSYGFDVWGTGTTVTVSSGGGGSGGGGSGGGGSDIVMTQSPASQSASLGESVTITCLASQTIGTWLAWYQQKPGKSPQFLIYAATSLADGVPSRFSGSGSGTKFSFKISSLQAEDFVSYYCQQLYSTPFTFGGGTKLEIKGSAGSAAGSGEFAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKGSAGSAAGSGEFQVQLVQSGGGVVQPGRSLRLSCSSSGYTFTRYTMHWVRQAPGKGLEWVAYINPSRGYTNYNQKFKDRFTISRDNSKNTLFLQMDSLRPEDTGVYFCARYYDDHYCLDYWGQGTPVTVSSGSSGSSSGTSSGGSSSSGSGDIQMTQSPSSLSASVGDRVTITCSASSSVSYMNWYQQTPGKAPKLLIYDTSKLASGVPSRFSGSGSGTDYTFTISSLQPEDIATYYCQQWSSNPFTFGQGTKLQITR

SEQ ID NO: 10 - DNA, 2256 bp, synthetic construct
  source 1..2256: mol_type = other DNA
atgtggtggcgactttggtggctgctgttgctgctgcttctgctgtggcctatggtctgggcccaagtccagctgcaagagtctggcacagaactggttaagccaggcgccagcgtgaagctgtcttgtaaagccagcggctacaccttcaccagctactggatgcactggatgaagcagaggcctggacagggactcgagtggatcggcaacatcaatcctagcaacggcggcaccaactacaacgagaagttcaagaacaaggccacactgaccgtggacaagagcagctctacagcctacatgcagctgagcagcctgacaagcgaggacagcgccgtgtactactgcgccagaagagactactactacggcagcagctacggcttcgatgtgtggggaactggcaccaccgtgacagtttctagcggaggcggaggaagtggtggcggaggttctggcggcggaggatctgatatcgtgatgacacagagccctgccagccagtctgcctctctgggagagtctgtgaccatcacatgtctggccagccagaccattggaacatggctggcctggtatcagcagaaacctggcaagagccctcagttcctgatctacgccgccacatctctggctgatggcgtgccaagcagattttctggctctggcagcggcacaaagttcagcttcaagatcagctccctgcaggccgaggacttcgtgtcctactattgccagcagctgtactctacccctttcacctttggcggaggcaccaagctggaaatcaagggctctgctggatctgccgccggatctggcgaatttgctcctcctgttgccggacctagcgtgttcctgtttcctccaaagcctaaggacaccctgatgatcagcaggacccctgaagtgacctgtgtggtggtggatgtgtcccacgaggatcccgaggtgcagttcaattggtacgtggacggcgtggaagtgcacaacgccaagaccaagcctagagaggaacagttcaacagcaccttcagagtggtgtccgtgctgacagtggtgcatcaggactggctgaacggcaaagagtacaagtgcaaggtgtccaacaagggcctgcctgctcctatcgagaaaaccatcagcaagacaaagggccagcctcgcgagcctcaggtttacacactgcctccaagccgggaagagatgaccaagaatcaggtgtccctgacctgcctggtcaagggcttctaccctagcgacatcagcgtggaatgggagagcaatggccagcctgagaacaactacaagaccacacctcctatgctggacagcgacggctcattcttcctgtacagcaagctgactgtggataagtcccggtggcagcagggcaatgtgttcagctgttctgtgatgcacgaggccctgcacaaccactacacccagaagtctctgtctctgagccctggcaagggaagcgcaggttctgctgctggaagcggagaatttcaggtgcagctggttcagtcaggcggaggtgttgtgcagcctggcagaagcctgagactgagctgtagcagctccggatacacattcacccggtacaccatgcactgggttcgacaggcccctggcaaaggactggaatgggtcgcctacatcaaccccagccggggctacaccaattataaccagaagtttaaggaccggttcaccatcagccgggacaacagcaagaataccctgttcctgcagatggacagcctgcggcctgaagataccggcgtgtacttttgcgcccggtactacgacgaccactactgcctggattattggggccagggcacacctgtgaccgtgtccagtggtagcagtggctctagctctggaacaagcagcggcggatcttcttctagcggctccggcgatatccagatgacccagtctccaagctctctgagcgcctctgtgggcgacagagtgacaattacatgtagcgccagctccagcgtgtcctatatgaattggtatcaacagaccccaggcaaggccccaaagctgctgatctatgatacctccaagctggcctccggcgtgccatctagattttccggaagcggcagcggaaccgattacacgttcaccattagcagcctgcagccagaggacattgccacctattactgccagcagtggtccagcaatcccttcaccttcggccaggggaccaagctgcagatcaccagataa

SEQ ID NO: 11 - protein, 21 aa, synthetic construct
MWWRLWWLLLLLLLLWPMVWA

SEQ ID NO: 12 - DNA, 63 bp, synthetic construct
  source 1..63: mol_type = other DNA
atgtggtggcgcctgtggtggctgctgctgctgctgctgctgctgtggcccatggtgtgggcc

SEQ ID NO: 13 - DNA, 405 bp, synthetic construct
  source 1..405: mol_type = other DNA
gcacaggtgcagctgcaggagtcgggcccaggactggtgaggccttcggggaccttgtccctgacctgcgcagtgtcaggtgactccatcagcggtggaaattggtggagttgggtccggcaggccccagggaaggggctggaatggattggggaaattcatcatagtgggaacacaaactacaacccggccctcgagagtcgactcaccatgtcagtcgacaagtccaagaatcagttctccctgactctgacctctgtgaccgccgcggacgcggccgtgtatttttgtgcgagagataccgaggagggactagaagtgcctggtttggagtacggtatggacgtctggggccaggggaccacagtctccgtctcctcagcatccccgaccagccccaaggtc

SEQ ID NO: 14 - DNA, 60 bp, synthetic construct
  source 1..60: mol_type = other DNA
ggcagcagcggcagcagcagcggtaccagcagcggcggcagcagcagcagcggcagcggc

SEQ ID NO: 15 - DNA, 321 bp, synthetic construct
  source 1..321: mol_type = other DNA
gagctcgtgatgactcagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccccctcactttcggcggagggaccaaggtggaaatcaaa

SEQ ID NO: 16 - DNA, 36 bp, synthetic construct
  source 1..36: mol_type = other DNA
ggaagcgcgggctccgccgcaggcagcggggagttc

SEQ ID NO: 17 - DNA, 648 bp, synthetic construct
  source 1..648: mol_type = other DNA
gcaccacccgtcgctggtcctagcgttttcctgttcccacctaagcccaaggacactctcatgatctcgaggaccccagaagtgacttgcgtagttgtcgacgtcagtcatgaggacccagaagttcagttcaactggtacgtggacggcgtggaagtccacaatgctaagacaaagccccgagaagaacagtttaactcaacgtttagagtggtgagtgttctgacagtggtgcaccaggattggctcaatgggaaagagtacaagtgcaaggtgagcaacaaaggcctgccggcccctatagaaaaaaccattagcaagacaaagggtcagcctagagagccacaggtgtataccctccccccttctcgtgaggagatgaccaaaaaccaggtatctttaacatgcctagtcaagggcttctatccctcagatatctccgtggaatgggagagtaacggccagcccgagaataattacaaaactaccccgcctatgctggatagcgatggtagcttctttttgtactccaaactgacggtggacaagtctcggtggcaacaaggaaatgttttttcatgttcagtcatgcacgaggccctgcacaaccattacacccagaagtctttgagtttaagcccaggcaaa

SEQ ID NO: 18 - DNA, 227 bp, synthetic construct
  source 1..227: mol_type = other DNA
gcaccacccgtcgctggtcctagcgttttcctgttcccacctaagcccaaggacactctcatgatctcgaggaccccagaagtgacttgcgtagttgtcgacgtcagtcatgaggacccagaagttcagttcaactggtacgtggacggcgtggaagtccacaatgctaagacaaagccccgagaagaacagtttaactcaacgtttagagtggtgagtgttctgac

SEQ ID NO: 19 - DNA, 421 bp, synthetic construct
  source 1..421: mol_type = other DNA
agtggtgcaccaggattggctcaatgggaaagagtacaagtgcaaggtgagcaacaaaggcctgccggcccctatagaaaaaaccattagcaagacaaagggtcagcctagagagccacaggtgtataccctccccccttctcgtgaggagatgaccaaaaaccaggtatctttaacatgcctagtcaagggcttctatccctcagatatctccgtggaatgggagagtaacggccagcccgagaataattacaaaactaccccgcctatgctggatagcgatggtagcttctttttgtactccaaactgacggtggacaagtctcggtggcaacaaggaaatgttttttcatgttcagtcatgcacgaggccctgcacaaccattacacccagaagtctttgagtttaagcccaggcaaa

SEQ ID NO: 20 - DNA, 36 bp, synthetic construct; identical to SEQ ID NO: 16
  source 1..36: mol_type = other DNA

SEQ ID NO: 21 - DNA, 357 bp, synthetic construct
  source 1..357: mol_type = other DNA
caggtccagcttgtgcagtccggcggcggagtcgttcaaccaggaagatctttgcgattaagttgcagctctagtgggtacacattcacaagatatacgatgcattgggttcggcaggctcccggtaaaggactggaatgggtggcatacattaacccgtccaggggctatactaactacaaccaaaagtttaaggaccggttcacaatcagccgtgacaattcaaaaaataccctcttcctgcagatggattcactccgccccgaggataccggggtgtacttttgcgccaggtactacgacgaccactattgtctggattattgggggcagggtacccctgtgactgtaagctcc

SEQ ID NO: 22 - DNA, 60 bp, synthetic construct; identical to SEQ ID NO: 14
  source 1..60: mol_type = other DNA

SEQ ID NO: 23 - DNA, 321 bp, synthetic construct
  source 1..321: mol_type = other DNA
gacattcagatgactcagagcccaagttcgctgtcagcaagcgtgggcgacagagtaacaattacctgttccgcctcctcaagtgtgtcttacatgaattggtatcagcagactcccgggaaggcgcccaaactattgatctacgataccagcaaactggctagcggggtcccatcaaggttcagtggcagcggttccggaacggactatacctttaccataagctccctgcagcctgaggatatcgccacatactactgccagcagtggtcttctaacccgttcacttttggacaaggcacaaagctccaaatcacacgg

SEQ ID NO: 24 - protein, 20 aa, synthetic construct
GSSGSSSGTSSGGSSSSGSG

SEQ ID NO: 25 - protein, 109 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 26 - protein, 107 aa, synthetic construct
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK